(19) United States
(12) Patent Application Publication
(10) Pub. No.: US 2026/0010933 A1
(43) Pub. Date: Jan. 8, 2026

(54) Customer Feedback Management System

(71) Applicant: Mickaël A. Sebban, North Miami Beach, FL (US)

(72) Inventor: Mickaël A. Sebban, North Miami Beach, FL (US)

(21) Appl. No.: 19/257,692

(22) Filed: Jul. 2, 2025

Related U.S. Application Data
(60) Provisional application No. 63/667,849, filed on Jul. 5, 2024.

(51) Int. Cl. G06Q 30/0282; A61B 5/0533; G06F 16/22; G06Q 20/20; G06Q 30/0238

(52) U.S. Cl. CPC G06Q 30/0282; A61B 5/0533; G06F 16/22; G06Q 20/202; G06Q 30/0238

(57) ABSTRACT

A system for managing customer feedback includes a first computer; a second computer; a server; a database; the first computer configured to obtain user feedback and configured to transmit the user feedback to the server, the database, and the second computer; software executing on the server to determine whether the user feedback meets a threshold, the server configured to transmit the user feedback to the database and the second computer; software executing on the server to prevent transmission of the user feedback to the database if the user feedback does not meet the threshold; and software executing on the server to display an alert on the second computer if the customer feedback does not meet said threshold.

### TECHNICAL FIELD

The present application relates to feedback collection, management, and utilization. More particularly, the present application relates to methods for gathering, analyzing, and responding to customer feedback primarily for brick-and-mortar businesses.

### BACKGROUND

The landscape of customer feedback management is dramatically different between traditional brick-and-mortar venues and technology-driven corporations. Legacy venues often struggle to adapt to rapid technological changes due to entrenched mindsets and operational inertia. This lag contrasts sharply with tech-savvy corporations that are early adopters and digital leaders who leverage cutting-edge technologies to enhance customer engagement and satisfaction.

Brick-and-mortar businesses typically rely on physical customer interactions and may need more immediate mechanisms for efficiently capturing and analyzing customer feedback. Without real-time data, these venues respond slower to customer needs and often do not address negative feedback. As a result, a gap occurs in customer service, negatively impacting reputation and competitiveness.

In contrast, technology corporations use digital platforms to integrate customer feedback directly into their service delivery.

The transition from traditional methods to such innovative feedback systems can be challenging for many brick-and-mortar venues due to the need for technological integration and a shift in mindset from reactive to proactive customer service. However, those who choose to leap forward with the adoption of these technologies, with the help of our system, can significantly enhance their ability to compete in an increasingly digital marketplace, closing the gap with their tech-forward counterparts.

### SUMMARY

The present invention introduces a sophisticated system designed to radically enhance customer feedback management, collection, and utilization, particularly tailored for brick-and-mortar establishments. By leveraging cutting-edge technologies such as near-field communication (“NFC”), radio frequency identification (“RFID”), quick response (“QR”) codes, blockchain, and artificial intelligence, including natural language processing (NLP), the present invention offers a comprehensive solution that bridges the gap between traditional customer service practices and modern technological capabilities.

The proposed system represents a shift towards more agile and responsive customer service methods. This system enables immediate feedback collection at the point of interaction, providing real-time opportunities to address issues before they escalate to the point of no return. The system allows businesses to preemptively manage their online reputation by filtering and curating feedback, thus avoiding the immediate impact of negative reviews on platforms like Google.

A system according to the present teachings may include, but is not limited to, a first computer, a second computer, a server, a database, said server configured to obtain user feedback and configured to transmit said user feedback to said server, said database, and said second computer, software executing on said server to determine whether said user feedback meets a threshold, said server configured to transmit said user feedback to said database and said second computer, software executing on said server to prevent transmission of said user feedback to said database if said user feedback does not meet said threshold, and software executing on said server to display an alert on said second computer if said customer feedback does not meet said threshold.

A method according to the present teachings may include, but is not limited to, providing a system that includes a first computer, a second computer in communication with the first computer, a server in communication with the first and second computers, and a database in communication with the first and second computers, acquiring, at the first computer, feedback from a user, transmitting said feedback to the server, analyzing, via software executing on the server, said feedback and comparing said feedback to a threshold, in response to said feedback meeting said threshold, transmitting via the server said feedback to the database for collection, wherein the database is accessible by a third-party device for viewing, in response to said feedback failing to meet said threshold, issuing via the server an alert to the second computer regarding said feedback failing to meet said threshold.

Systems and methods according to the present teachings also include an “Internal Review Processing Center” (IRPC), which acts as a buffer, preventing a ‘fait accompli,’ by intercepting and managing reviews rated at or below a threshold set by a user. Businesses are then given a second chance to engage in remediation efforts, possibly offering compensation to dissatisfied customers, which can transform a potentially negative experience into a positive one. This approach not only improves customer satisfaction but also aids in retaining them by showing a willingness to resolve issues promptly.

Opt-in review comments may be stored in a database.

This system promotes and enhances customer feedback management for offline venues through the use of machine-readable identifiers or tags such as RFID, NFC, or other equivalent technologies to gather customer reviews directly. It allows for immediate feedback collection, real-time response to customer dissatisfaction, and a mechanism to improve online ratings by filtering out (while the curation process takes place) negative reviews before they reach platforms like Google. Venues will want to amass as many reviews as possible while being given a chance to address negative user experiences before they impact determining metrics.

Tags are manually or batch-scanned for platform provisioning purposes. A specific device reader or even a phone, such as Android with an NFC Adapter class or iOS Core NFC, may be used for that purpose.

Electronic tags include, but are not limited to, Near Field Communication (NFC) tags, Radio-Frequency Identification (RFID) tags, and Quick Response (QR) code carrying a unique Identifier (UID). The NFC tag may be embedded in a wristband or in a card which is given to an employee or other representative of the venue.

The electronic tags take the customer (end-user) to a service platform for an interactive process to review the service received or the job performed by the venue. The customer (end-user) may be invited to access an NFC tag embedded in a wristband or card borne by the employee or other representative, for example by the bearer of the NFC tag extending the card or wristband toward the customer (end-user) who would interact with said NFC tag using their phone or device reader. The owner can access a backend dashboard from which all the reviews are presented and handled.

Reviews at or above a certain threshold (e.g., five-stars) are left to flow towards the platform, receiving the rating and comments while providing discretionary data to our system's database. Venue owners can access insights, metrics, and information that help with loyalty-emboldening initiatives via actionable. The flow-through of reviews at or above the threshold may be automatic, or the customer (end-user) may be prompted to re-enter the same review, or the customer (end-user) may be notified by the system that the contents of the review has been automatically added to their device's clipboard (e.g., automatic copy) and prompted to paste the contents into a third-party review database (e.g., Google Reviews) after the customer (end-user) is automatically redirected to the respective venue's landing page or site on the third-party review database.

Impactful bad reviews (e.g., four-stars and below) are intercepted before reaching their destination, allowing a second chance for curation initiatives.

On-premises clients are motivated to share immediate feedback through multiple mediums, with instant gratification incentives (e.g., “token-based” rewards), ease of access, and possibly real-time resolution. This can significantly improve customer experience and foster a strong emotional connection with the venue. Customers may use discretionary anonymous identity with blockchain zero-knowledge proofs (ZKP).

### BRIEF DESCRIPTION OF THE DRAWINGS

FIG. 1 presents one embodiment of a system according to the present teachings.

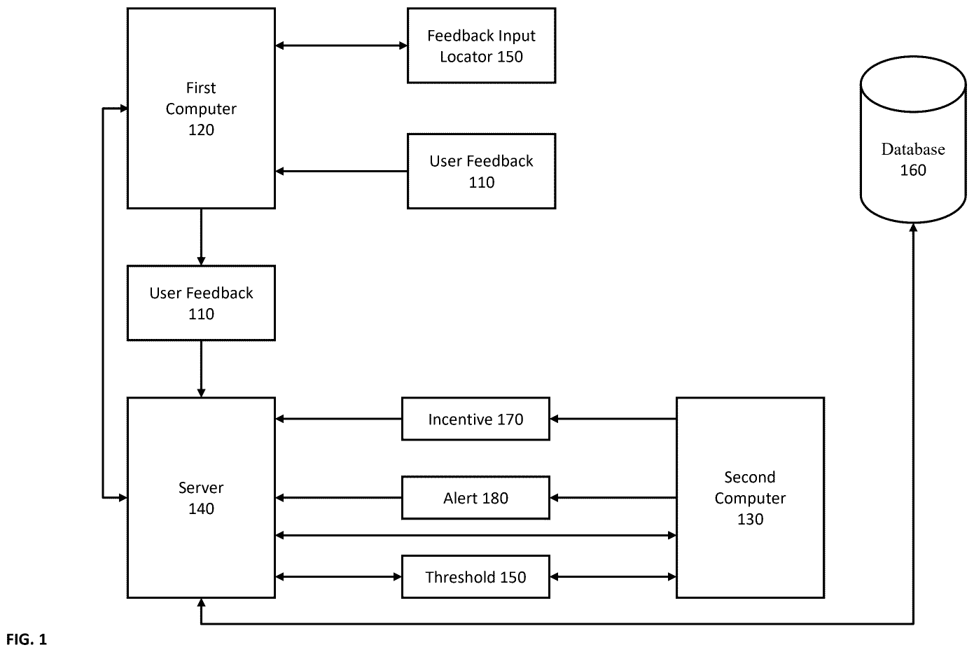

FIG. 2 presents the system of FIG. 1 with additional components according to the present teachings.

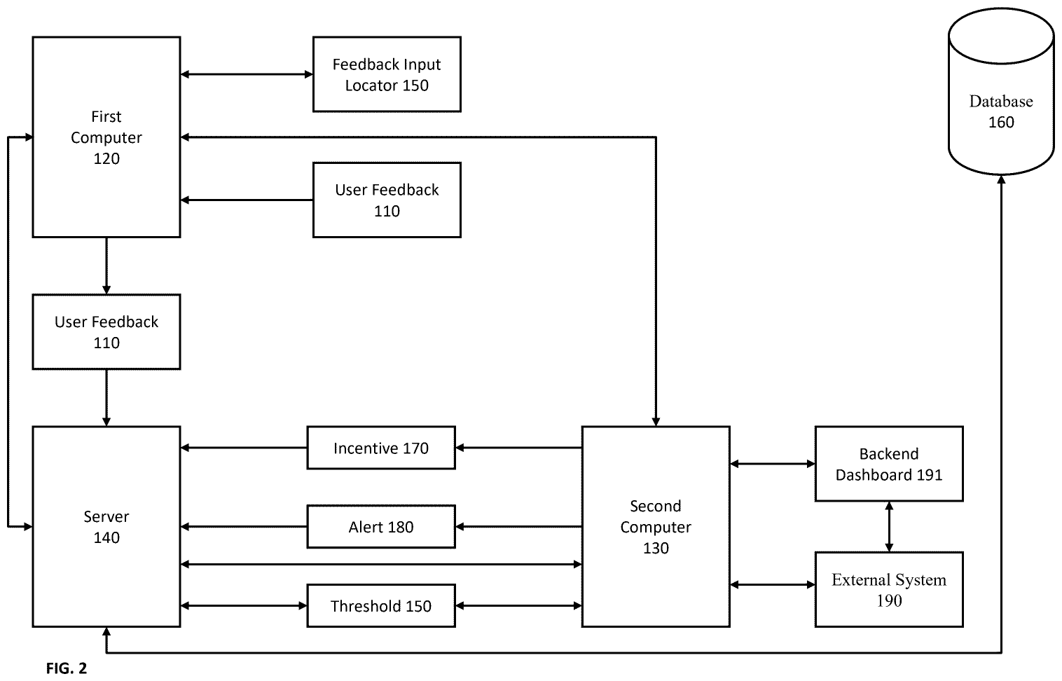

FIG. 3 presents one embodiment of a method according to the present teachings.

FIG. 4 presents the method of FIG. 3 with additional steps according to the present teachings.

### DETAILED DESCRIPTION

The present teachings are described more fully hereinafter with reference to the accompanying drawings. The following description is presented for illustrative purposes only and the present teachings should not be limited to these embodiments. Any computer configuration and architecture satisfying the speed and interface requirements herein described may be suitable for implementing the system and method of the present embodiments. The present invention is an advanced system designed to elevate customer feedback collection, management, and utilization, particularly for brick-and-mortar businesses. It is further understood that the terms “customer” and “user” are meant to be synonymous when used herein. This comprehensive description will outline the primary components and functionalities underpinning this innovative system's operation.

In compliance with the statute, the present teachings have been described in language more or less specific as to structural and methodical features. It is to be understood, however, that the present teachings are not limited to the specific features shown and described, since the systems and methods herein disclosed comprise preferred forms of putting the present teachings into effect.

For purposes of explanation and not limitation, specific details are set forth such as particular architectures, interfaces, techniques, etc. in order to provide a thorough understanding. In other instances, detailed descriptions of well-known devices, circuits, and methods are omitted so as not to obscure the description with unnecessary detail.

Generally, all terms used in the claims are to be interpreted according to their ordinary meaning in the technical field, unless explicitly defined otherwise herein. All references to a/an/the element, apparatus, component, means, step, etc. are to be interpreted openly as referring to at least one instance of the element, apparatus, component, means, step, etc., unless explicitly stated otherwise. The steps of any method disclosed herein do not have to be performed in the exact order disclosed, unless explicitly stated. The use of “first”, “second,” etc., for different features/components of the present disclosure are only intended to distinguish the features/components from other similar features/components and not to impart any order or hierarchy to the features/components.

Fundamentally, the present teachings are based on a collection of customer feedback 110 during or after said customer's use of an establishment's services. The customer may be an individual, a group of individuals, or an entity. Many possible establishments are envisioned, including, without limitation, restaurants, movie theaters, and bowling alleys. Customer feedback 110 is generally understood to be in electronic form (e.g., through a survey), but other means are envisioned, including oral feedback. Customer feedback is preferably obtained while the customer is physically present at an establishment, such that the establishment may have an opportunity to remediate if necessary. Customer feedback is preferably obtained on a first computer 120, which may comprise a phone, tablet, personal computer, or other device capable of communication with a feedback input locator 150.

The present invention may employ one or more of various technologies, including NFC, RFID, and QR codes, integrated into user interfaces such as smartphones, tablets, or kiosks, for the purpose of obtaining customer feedback. These one or more various technologies are referred to throughout as the feedback input locator 150. It is envisioned that the one or more various technologies 150 may be in a physical form (e.g., QR code on a card placed on a table) or an electronic form (e.g., integrated into an app downloaded by the customer, integrated into a tablet present at the brick-and-mortar establishment). This setup enables customers to submit feedback 110 directly at various interaction points within a business setting. For example, a restaurant customer may submit feedback 110 after being seated, prior to payment but after finishing their meal, and/or after paying for their food, among other times. It is envisioned that the first computer 120 may be in communication with the feedback input locator 150 at various times throughout the user's patronage of the establishment.

Additionally, a system according to the present teachings may be capable of collecting biofeedback using devices like GSR (Galvanic Skin Response) sensors that measure physiological responses, providing deeper insights into the emotional impacts of customer experiences. It is understood herein that customer feedback 110 may comprise, in whole or in part, biofeedback. These data are crucial for understanding subtle customer reactions that textual feedback might not explicitly state.

A central feature of the present invention is the Internal Review Processing Center (herein “IRPC”), which functions as a sophisticated filter for managing and categorizing customer reviews based on their ratings. The IRPC comprises a first computer 120, a second computer 130, and a server 140. After the user feedback 110 is inputted into the first computer 120, said first computer 120 transmits said user feedback 110 to the server 140. The server 140 may store user feedback 110 in all formats. Following transmission, the server 140 compares the user feedback 110 to a threshold 150. If the user feedback 110 is greater than the threshold 150, the server 140 may transmit the user feedback 110 to a database 160. Once the feedback 110 is transmitted to the database 160, it may be published on said database 160 for viewing by one or more third parties.

In alternative embodiments, the first computer 120 may transmit the user feedback 110 to the second computer 130, rather than to the server 140.

It is envisioned that the threshold 150 may be modifiable by an operator, which may include a business proprietor. Examples of operators as understood include restaurant owners. The threshold 150 may comprise one or more metrics. Said metrics may be quantitative or qualitative. If a metric is quantitative, it might be in any form meant to convey quality or reputation to a third party. Examples of a quantitative metric include, but are not limited to, rankings based on numbers of “stars” (e.g., a satisfied customer may provide a “five-star” ranking) and rankings based on a number alone (e.g., a ranking on a scale of 1 to 100, with 100 indicating a highly satisfactory experience).

Other metrics envisioned for a comparison to the threshold 150 may be qualitative. For qualitative metrics, the IRPC may utilize artificial intelligence (herein “AI”) and/or natural language processing (herein “NLP”) to analyze text-based user feedback 110, extract useful information, and/or determine sentiment and/or intent from customer feedback 110. This allows for sophisticated processing and categorization of feedback to derive actionable insights.

NLP techniques encompassed by the present teachings may include, but are not limited to, term frequency-inverse document frequency (herein “TF-IDF”). TF-IDF evaluates the importance of a word within a collection of documents or feedback corpus relative to its frequency. In the context of customer feedback, TF-IDF helps distinguish words that are frequently used in negative or positive contexts across multiple reviews, highlighting potential areas of concern or success that might require the business's attention.

NLP techniques may also comprise topic modeling. Topic modeling is employed as a method to uncover abstract topics within large volumes of text and identifies predominant themes or patterns in customer reviews. This method allows businesses to discern overarching issues that resonate with customers, facilitating targeted improvements and tailored responses.

Reviews with higher ratings may be automatically processed and published to promote the business's public profile. Conversely, lower-rated feedback is intercepted, allowing the business to address issues in real time. This dynamic filtering mechanism is instrumental in converting potentially negative experiences into positive outcomes, thereby safeguarding and enhancing the business's reputation.

After the user feedback 110 is analyzed using the IRPC, it is either transmitted from the server 140 to the database 160 for publication or some other action is taken as configured by an operator. Preferably, the user feedback 110 is only transmitted from the server 140 to the database 160 if said user feedback 110 meets or exceeds the threshold 150. If the user feedback 110 does not meet the threshold 150, the server may transmit a message to the second computer 130. In response, or at any time, the second computer 130 may transmit an alert 180 to the server 140 for transmission to the first computer 120. In some embodiments, the second computer may transmit an alert 180 or other message to the first computer 120.

It is also envisioned that messages may be automatically transmitted from the server 140 to the first computer 120 if the user feedback 110 meets or exceeds the threshold 150. Messages may be automatically transmitted from the server 140 to the first computer 120 if the user feedback 110 does not meet the threshold 150. Examples of messages transmitted based on the contents of the user feedback 110 include, but are not limited to, loyalty vouchers, “thank you” messages, “disappointment” messages, and prompts to sign up for loyalty programs.

Some embodiments of a system or method according to the present teachings may incorporate a rewards system or other compensation program to engage customers further and encourage honest, detailed feedback. Customers providing feedback 110 may be compensated with digital tokens, which can be redeemed for various services or products, fostering continued engagement and enhancing customer loyalty.

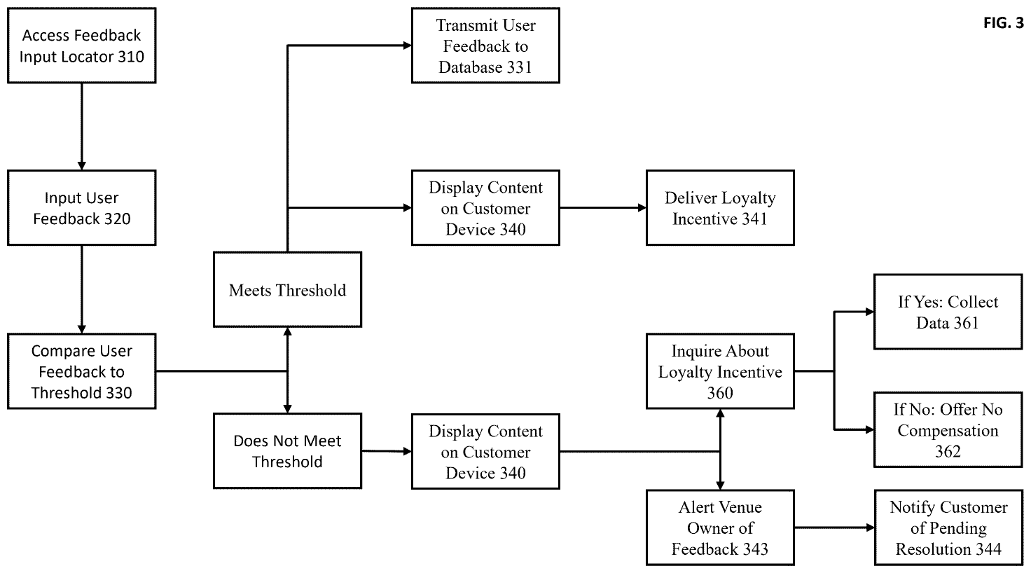

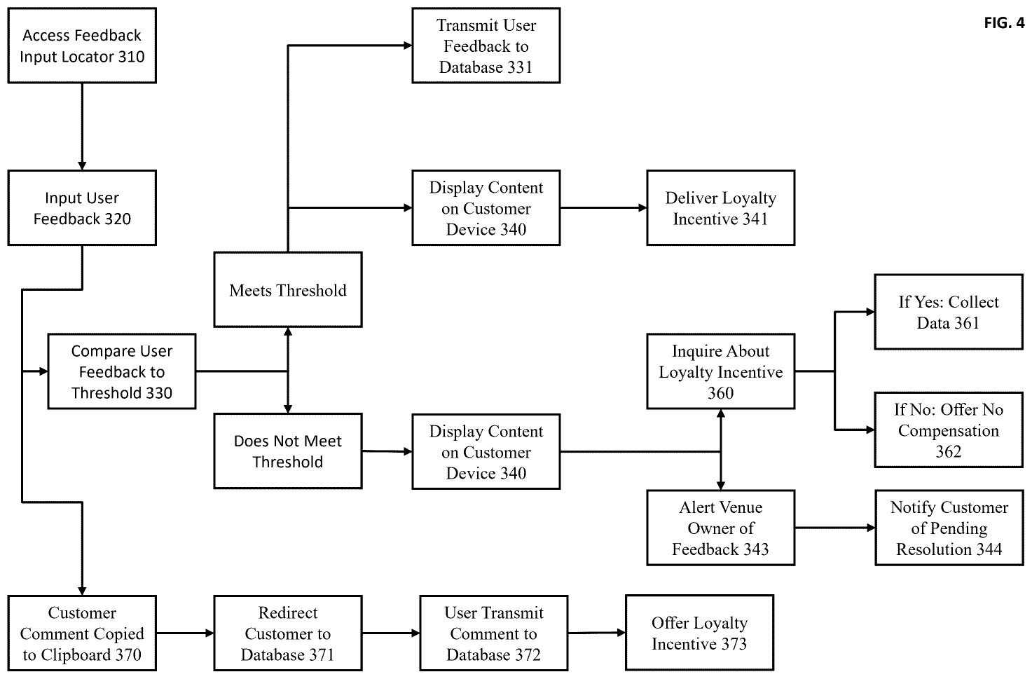

The present teachings are not solely directed to systems for customer feedback management but also include methods for the same. FIGS. 3 and 4 provide exemplary embodiments of said methods.

Other features for the present technology are envisioned, including features meant to address privacy concerns, both from a user's and a proprietor's point of view. Advanced applications of blockchain technology in the present invention may ensure transparency and trust in the review process, where customer reviews and business responses are immutably recorded. This may also include the use of zero-knowledge proofs to maintain user privacy while verifying the legitimacy of transactions and feedback.

Other privacy features may include an ‘anonymity status’ option, whereby customers may choose to have their identity remain secret. This option can be presented at the time of providing feedback or at other times. This option may include sub-options, including choices for a user not to display their personal information (e.g., name, location) to the restaurant or for transmission to the database. This option may significantly reduce the fear of retribution, offering a secure and confidential channel for customers to share genuine feedback. This practice enhances the public image of businesses by demonstrating a commitment to excellence and transparency. The integration of the IRPC allows for effective management of lower-rated reviews, turning potential negatives into opportunities for improvement. Furthermore, blockchain technology guarantees the authenticity and privacy of the feedback process, while the cryptocurrency rewards system motivates customers to engage actively. This holistic approach enhances customer satisfaction and loyalty and empowers businesses to continuously refine their services, thereby maintaining a competitive edge in the digital marketplace.

Embodiments of the present invention may utilize real-time customer feedback analysis and/or statistical measuring with topic modeling and term frequency-inverse document frequency. Real-time customer feedback analysis may provide a proprietor with real-time analytics, performance metrics, and the ability to manage and respond to customer feedback efficiently. This dashboard is vital for venue owners and managers to monitor and improve customer satisfaction and operational performance continually.

Other features envisioned for the present technology include a backend dashboard 191 for accessing user feedback 110 and/or for communicating with an external system 190. The external system 190 may include a point of sale (herein “POS”) system or other software. The backend dashboard 191 may provide business user options that display the user feedback 110 collected from customers based on date, time, frequency of patronage, and more.

While the present teachings have been described above in terms of specific embodiments, it is to be understood that they are not limited to those disclosed embodiments. Many modifications and other embodiments will come to mind to those skilled in the art to which this pertains, and which are intended to be and are covered by both this disclosure and the appended claims. For example, in some instances, one or more features disclosed in connection with one embodiment can be used alone or in combination with one or more features of one or more other embodiments. It is intended that the scope of the present teachings should be determined by proper interpretation and construction of any claims and their legal equivalents, as understood by those of skill in the art relying upon the disclosure in this specification and the attached drawings.

1. A system for managing customer feedback, comprising:
a first computer;
a second computer;
a server;
a database;
said first computer configured to obtain user feedback and configured to transmit said user feedback to said server, said database, and said second computer;
software executing on said server to determine whether said user feedback meets a threshold, said server configured to transmit said user feedback to said database and said second computer;
software executing on said server to prevent transmission of said user feedback to said database if said user feedback does not meet said threshold; and
software executing on said server to display an alert on said second computer if said customer feedback does not meet said threshold.

2. The system of claim 1, wherein the first computer comprises a smartphone or tablet computer.

3. The system of claim 1, wherein the server stores a local copy of the user feedback.

4. The system of claim 1, wherein the second computer is configured to receive operator input modifying the threshold.

5. The system of claim 4, wherein the threshold is a quantitative metric.

6. The system of claim 4, wherein the threshold is qualitative and wherein the software executing on said first computer for determining whether said user feedback meets said threshold utilizes artificial intelligence and/or natural language processing to determine whether said user feedback meets said threshold.

7. The system of claim 1, wherein said user feedback is assigned an identifier using a zero-knowledge proof, and wherein said database displays said user feedback using said identifier.

8. The system of claim 1, wherein the first computer is configured to receive location data from at least one of an RFID, NFC, or QR code, wherein the location data provides a URL through which user feedback is input.

9. The system of claim 1, wherein the software executing on the second computer is in electronic communication with point-of-sale software.

10. The system of claim 1, further comprising software executing on said first computer to provide a message offering an incentive to a user if said user feedback does not meet said threshold.

11. The system of claim 1, wherein the database collects and organizes at least a portion of said user feedback, and wherein the database is accessible by a third party device for displaying said at least a portion of said user feedback.

12. A system for managing customer feedback, comprising:
a first computer;
a second computer;
a database;
software executing on said first computer to receive multiple user feedback inputs;
software executing on said server to determine whether each user feedback input meets a threshold;
software executing on said server to transmit user feedback inputs that meet the threshold to said database, and prevent transmission of user feedback inputs that fail in meeting the threshold to said database, wherein said software issues a notification to said second computer indicating the prevention of transmission of said user feedback inputs that fail in meeting the threshold;
wherein said database collects said user feedback inputs that meet the threshold.

13. The system of claim 11, wherein the first computer is in communication with a galvanic skin response device and said first computer is configured to receive user feedback in the form of biofeedback.

14. The system of claim 11, wherein said first computer comprises a smartphone that receives said user feedback inputs via an application.

15. A method for managing customer feedback, comprising:
providing a system that includes a first computer, a second computer in communication with the first computer, a sever in communication with the first and second computers, and a database in communication with the first and second computers;
acquiring, at the first computer, feedback from a user;
transmitting said feedback to the server;
analyzing, via software executing on the server, said feedback and comparing said feedback to a threshold;
in response to said feedback meeting said threshold, transmitting via the server said feedback to the database for collection, wherein the database is accessible by a third-party device for viewing;
in response to said feedback failing to meet said threshold, issuing via the server an alert to the second computer regarding said feedback failing to meet said threshold.

16. The method of claim 15, wherein the customer device comprises one or more of a smartphone or tablet computer.

17. The method of claim 15, wherein the second computer comprises one or more of a smartphone or tablet computer.

18. The method of claim 15, further comprising:
in response to said feedback failing to meet said threshold, transmitting via the first computer a notice offering an incentive to said customer device.
